(19) United States
(12) Patent Application Publication
(10) Pub. No.: US 2017/0239030 A1
(43) Pub. Date: Aug. 24, 2017

(54) ARTIFICIAL VAGINA

(71) Applicant: Bruce Eilts, Baton Rouge, LA (US)

(72) Inventor: Bruce Eilts, Baton Rouge, LA (US)

(21) Appl. No.: 15/052,360

(22) Filed: Feb. 24, 2016

(51) Int. Cl. A61D 19/02; A61M 39/24; A61F 7/00; A61F 5/453

(52) U.S. Cl. CPC A61D 19/021; A61F 5/453; A61M 39/24; A61F 7/007; A61M 2205/3368

(57) ABSTRACT

Artificial vaginas are disclosed that include an elongate housing that may be cylindrical, a liner within the elongate housing that is attached to the elongate housing, a volume of pressurized warm air between the elongate housing and the liner, a pressure relief valve, an aperture centrally located within the elongate housing and the liner and an elongate body. The elongate body may contain a heating element, may be arranged to substantially fill the aperture and may be positioned within the aperture to prepare the artificial vagina for use.

Artificial vaginas described herein may be used as part of artificial insemination of animals. Artificial vaginas described herein may be particularly useful in equine artificial insemination. Certain artificial vaginas disclosed herein may be lightweight, may provide temperature control and may provide pressure control.

### BRIEF DESCRIPTION OF THE DRAWINGS

FIG. 1 shows an elevation view of the artificial vagina.

FIG. 2 shows a section view of the artificial vagina on a heating stand.

### DETAILED DESCRIPTION

### Example 1

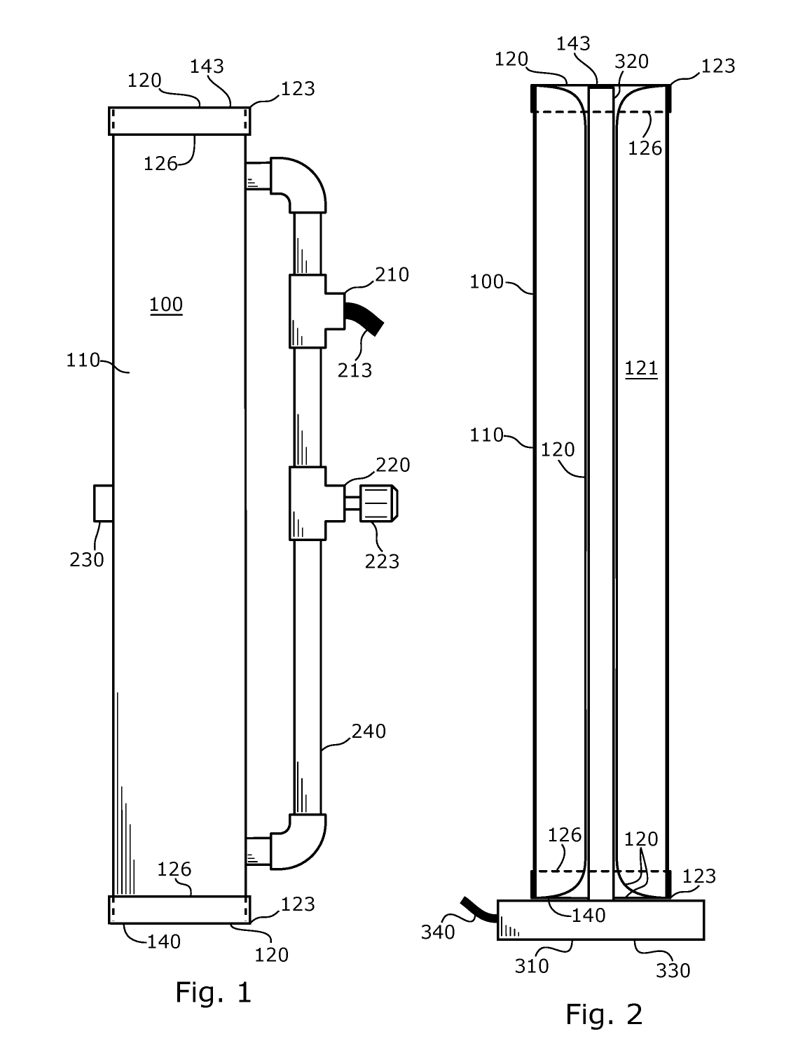

FIG. 1 depicts an elevation view of the artificial vagina. Shown in FIG. 1 are: Artificial vagina 100, Housing 110, Liner 120, Flexible annular bag 121, Liner folds 123, Liner Ends 126, Entry end 140, Collection end 143, First Port 210, Pneumatic hose 213, Second Port 220, Relief valve 223, Third Port 230 and Handle 240. Artificial vagina 100 includes a Housing 110 and a Liner 120. Housing 110 may for example be a cylinder having a 4.5 inch internal diameter and 20 inch length. The housing length may be between 18 and 24 inches and in certain other examples may be between 14 and 36 inches. The housing internal diameter may be between 4 and 5 inches and in certain examples may be between 3.5 and 7.5 inches. Liner 120 is situated predominately within Housing 110 such that Flexible annular bag 121 is predominantly located within Housing 110. Liner folds 123 on each end of Housing 110 allow the Liner Ends 126 to be sealed against the exterior of Housing 110. Flexible annular bag 121 is the roughly annular shaped inflatable bag formation within Housing 110 which is formed by the joining of Housing 110 and Liner 120. Liner 120 is the flexible and inflatable portion of Flexible annular bag 121. Artificial vagina 100 may be configured to have an Entry end 140 and a Collection end 143 which may be interchangeable depending on the particular configuration of Artificial vagina 100. Entry end 140 and Collection end 143 may be designated by the use of Artificial vagina 100 such that Entry end 140 is the end that is penetrated by the male reproductive member and Collection end 143 is the end in which semen is collected. First Port 210 may, for example, be in fluid communication with Flexible annular bag 121 by way of Handle 240 such that Pneumatic hose 213 may be used to pressurize Flexible annular bag 121 to the operating pressure for Artificial vagina 100. Pneumatic hose 213 may alternatively connect to a manual or automatic pump which may take the form of a compressor, a blower or other device suitable for creating the appropriate operating pressure. Second Port 220 may be in fluid communication with both Relief valve 223 and Flexible annular bag 121. Relief valve 223 may be an adjustable pressure relief valve. Relief valve 223 may, for example, be set to 30 mmHg. The air introduced through First Port 210 may be preheated air or maybe air of ambient temperature. When collecting from an animal Flexible annular bag 121 may be inflated such that it substantially fills the space within Housing 110 or such that if fills a substantial portion of the space within Housing 110. Insertion of the male reproductive member into Housing 110 generally along the central axis of Housing 110 may cause a portion of the air in Flexible annular bag 121 to be expelled through Relief valve 223 thereby producing a desirable pressure and fit for the male reproductive member facilitating the collection of semen. Third Port 230 may be omitted, may be used for a pressure gauge to indicate the internal pressure of Flexible annular bag 121 or may be used as an exhaust port allowing the escape of air from Flexible annular bag 121. A variety of port locations may be used either on Handle 240 or positioned in other locations on Artificial vagina 100 and the location of those ports may be selected based on factors such as convenience, ease and evenness of heating among other factors.

FIG. 2 shows a section view of the body of artificial vagina on a heating stand. Depicted in FIG. 2 are: Artificial vagina 100, Housing 110, Liner 120, Liner folds 123, Liner Ends 126, Entry end 140, Collection end 143, Heating stand 310, Heating rod 320, Heater base 330 and Heater cord 340. The body of Artificial vagina 100 may be arranged and configured for placement on a Heating stand 310 allowing for both storage of Artificial vagina 100 and allowing for the warming of Liner 120 and the air within Flexible annular bag 121. In preparation for collection, the body of Artificial vagina 100 may be slid over Heating stand 310 such that the body of Artificial vagina 100 surrounds Heating rod 320. Heating rod 320 may have a nonstick surface or may be lubricated prior to the positioning of the body of Artificial vagina 100 on Heating stand 310. Flexible annular bag 121 may then be inflated such that Heating rod 320 is in contact with Liner 120. Heating rod 320 may then be activated thereby warming Liner 120 and the air within Flexible annular bag 121. As depicted in FIG. 1, Liner folds 123 secure Liner Ends 126 to the exterior of Housing 110 in a manner that creates a seal sufficient to allow Flexible annular bag 121 to be pressurized. The body of Artificial vagina 100 may rest on Heating stand 310 of Heater base 330 such that Entry end 140 is positioned adjacent to Heater base 330. Such a configuration may produce a temperature profile in Liner 120 that is more suited to collection, however Collection end 143 may also be positioned adjacent to Heater base 330. Heater cord 340 may be configured to provide power to Heating stand 310. Heating stand 310 may for example be configured to run off of a 120V AC power source, other AC power sources, a 12V DC power, source, or other DC power sources including batteries. Liner 120 and Housing 110 may be sized and configured for the collection of semen from a stallion.

### Example 2

The embodiments described in Example 1 may be used by first placing the body of Artificial vagina 100 on Heating stand 310 such that Liner folds 123 rests on Heater base 330. Relief valve 223 may be set to an operating pressure, a pressure slightly above the operating pressure, or an interim pressure that is less than the operating pressure. Flexible annular bag 121 may then be inflated by way of First Port 210 to an operating pressure or a pressure slightly above the operating pressure. A heating element or multiple heating elements within Heating rod 320 may then be engaged automatically, by a switch, by plugging in Heating stand 310 or by other appropriate means to heat Liner 120 to an operating temperature or to a temperature slightly above the operating temperature. The operating temperature may, for example, be 48° C. The body of Artificial vagina 100 may remain positioned on Heating stand 310 until it is ready for use. Heating stand 310 may be configured to provide a maintenance level of heating that is less than the heating required to bring Liner 120 to the operating temperature. The control of Heating rod 320 may be done with one or more thermometers adjacent to one or more of Liner 120 and Flexible annular bag 121 with the thermometer(s) being part of a control loop or control scheme. The control loop or control scheme may be controlled from within Heating stand 310 and may be set up to achieve and maintain Liner 120 at or near the operating temperature. When it is time for collecting, the pressure in Flexible annular bag 121 may be adjusted to the operating pressure either by adding air, for example, by way of Pneumatic hose 213 or by decreasing the pressure. A decrease in pressure may be achieved by altering the setting of Relief valve 223 or by either manually or automatically releasing some quantity of air through a valve. Use of Artificial vagina 100 may proceed in a manner comparable to the use of prior art water filled artificial vaginas however the version of Artificial vagina 100 depicted in FIG. 1 and various alternate embodiments may be capable of expelling air through Relief valve 223 thereby producing a desirable pressure and fit for the male reproductive member as described above. Commercially available inner liners may be used with Artificial vagina 100 and positioned within Liner 120.

### Example 3

Artificial vagina 100 may be configured such that it may be warmed for use through a hot air blower or pump. In such an embodiment, warm or hot air may be introduced through Second Port 220 and exhausted through Third Port 230 or introduced through Third Port 230 and exhausted through Second Port 220. A variety of alternate port configurations and flow paths may be employed. Configurations and flow paths that evenly distribute warm or hot air through Flexible annular bag 121 may provide a suitable heat distribution over the area of Liner 120. Thermometers including thermometers that make up part of an automatic control loop associated with the hot air blower or pump may be placed in proximity to Liner 120 such that the optimal temperature of Liner 120 may be achieved prior to use. In embodiments where a hot air blower or pump is used, the blower or pump may achieve the desired pressure or a secondary pump, which may be a manual pump, could be used to achieve the operating pressure. The Artificial vagina 100 depicted in FIGS. 1 and 2 of the drawings may be heated and pressurized to appropriate operating conditions with nearly any combination of the techniques described herein including combinations of techniques. For example, the Artificial vagina 100 may be heated simultaneously by a hot air blower and by a heating component such as Heating rod 320. It is further contemplated that a hot air blower or pump may be used that provides less than the operating pressure in which case a hand pump may be used to attain the operating pressure. Because the operating pressure is relatively low, a variety of pumps or blowers may be used to reach operating pressure including a hand operated bulb pump comparable to those used with blood pressure cuffs. The hot air blower or pump may be located in Heating stand 310, may be located on or adjacent to Housing 110, incorporated into the handle of Housing 110, or may be a separate component that temporarily placed in fluid communication with the Flexible annular bag 121. Similarly, the controller may be located in Heating stand 310, may be located on or adjacent to Housing 110, incorporated into the handle of Housing 110, or may be a separate component. Temperature control schemes may be directed to controlling the temperature ultimately experienced by the male reproductive member. To that end, the temperature control may be based on a measurement of liner temperature or by measuring the temperature of a component that typically has a temperature that is very close to the liner temperature. The proximity of Heating rod 320 to the portion of Liner 120 in contact with the male reproductive member makes heating by that method quick and effective. Some additional heating time may be required to ensure that the folds in Liner 120 not in contact with Heating rod 320 are brought to the desired temperature. In certain cases the temperature of the air within Flexible annular bag 121 may be what is measured for the control of temperature. In such cases adequate time should be allowed to ensure that Liner 120 reaches the desired temperature.

### Example 4

A large number of potential examples are consistent with the disclosures of the preceding examples. A significant portion of those potential examples may be characterized by the features of the example sets described below. Table 1 below describes 16 such groups of embodiments. For example, Example Set 4A describes a group of embodiments in which the relief valve pressure is at least 5 mmHg, the relief valve pressure is at most 45 mmHg, the Liner operating temperature is at least 40° C. and the housing length is at least 18 in.

TABLE 1

| | Relief valve | Relief valve | Liner operating | |
|---|---|---|---|---|
| | pressure | pressure | temperature | Housing length |
| Example | is at least | is at most | is at least | is at least |
| Set | (mmHg) | (mmHg) | (° C.) | (in) |
| 4 A | 5 | 45 | 40 | 18 |
| 4 B | 15 | 45 | 40 | 18 |
| 4 C | 5 | 60 | 40 | 18 |
| 4 D | 15 | 60 | 40 | 18 |
| 4 E | 5 | 45 | 35 | 18 |
| 4 F | 15 | 45 | 35 | 18 |
| 4 G | 5 | 60 | 35 | 18 |
| 4 H | 15 | 60 | 35 | 18 |
| 4 I | 5 | 45 | 40 | 14 |
| 4 J | 15 | 45 | 40 | 14 |
| 4 K | 5 | 60 | 40 | 14 |
| 4 L | 15 | 60 | 40 | 14 |
| 4 M | 5 | 45 | 35 | 14 |
| 4 N | 15 | 45 | 35 | 14 |
| 4 O | 5 | 60 | 35 | 14 |
| 4 P | 15 | 60 | 35 | 14 |

### Example 5

A large number of potential examples are consistent with the disclosures of the preceding examples. A significant portion of those potential examples may be characterized by the features of the example sets described below.

As used herein the “initial fill percent” is the percentage of the internal volume of Housing 110 occupied by Flexible annular bag 121 after removal from Heating stand 310 if Heating stand 310 or another similar heating method is used but before the insertion of the male reproductive organ. The initial fill percentage may, for example, be 80%. As used herein the “operational air expulsion percent” is the mass percentage of air expelled from Flexible annular bag 121 during the semen collection. The operational air expulsion percent may, for example, be 20% or 30%. The parameters set out in Table 2 may be used to characterize certain groups of embodiments of which certain versions of the embodiments depicted in FIGS. 1 and 2 are members and of which many other alternate embodiments are members. Table 2 below describes 16 such groups of embodiments. For example, Example Set 5A describes a group of embodiments in which the operating pressure is at least 10 mmHg, the housing internal diameter is at least 3.5 inches, the initial fill percent is at least 50% and the operational air expulsion percent is at least 5%. Each of Example Sets 5A-5P may share characteristics with any of Example Sets 4A-4P and each such combination may be descriptive of Examples 1-3 and descriptive of a subset of embodiments which may be practiced in combination with the other features and characteristics described herein. By way of example, the subset of Example Set 5A having the features of Example Set 4A describes a group of embodiments in which the relief valve pressure is at least 5 mmHg, the relief valve pressure is at most 45 mmHg, the liner operating temperature is at least 40° C., the housing length is at least 18 inches, the operating pressure is at least 10 mmHg, the housing internal diameter is at least 3.5 inches, the initial fill percent is at least 50% and the operational air expulsion percent is at least 5%.

TABLE 2

| | Operating | Housing internal | Initial fill | Operational air |
|---|---|---|---|---|
| | pressure | diameter | percent | expulsion percent |
| Example | is at least | is at least | is at least | is at least |
| Set | (mmHg) | (in) | (%) | (%) |
| 5 A | 10 | 3.5 | 50 | 5 |
| 5 B | 20 | 3.5 | 50 | 5 |
| 5 C | 10 | 4.0 | 50 | 5 |
| 5 D | 20 | 4.0 | 50 | 5 |
| 5 E | 10 | 3.5 | 70 | 5 |
| 5 F | 20 | 3.5 | 70 | 5 |
| 5 G | 10 | 4.0 | 70 | 5 |
| 5 H | 20 | 4.0 | 70 | 5 |
| 5 I | 10 | 3.5 | 50 | 10 |
| 5 J | 20 | 3.5 | 50 | 10 |
| 5 K | 10 | 4.0 | 50 | 10 |
| 5 L | 20 | 4.0 | 50 | 10 |
| 5 M | 10 | 3.5 | 70 | 10 |
| 5 N | 20 | 3.5 | 70 | 10 |
| 5 O | 10 | 4.0 | 70 | 10 |
| 5 P | 20 | 4.0 | 70 | 10 |

Because the artificial vagina will often be used shortly after the cessation of temperature and/or pressure control for the liner, the various individual components associated with temperature and pressure control may be located either on the body of Artificial vagina 100 or on Heating stand 310. However, in most embodiments, Relief valve 223 will be on Artificial vagina 100.

Artificial vaginas described herein may, for example, comprise: an elongate housing having a first opening and a second opening; a liner located within the elongate housing wherein the liner is attached to the elongate housing; a volume of air between the elongate housing and the liner wherein the volume of air is at least 35° C. and wherein the volume of air has a gauge pressure of at least 5 mmHg; an aperture centrally located within the elongate housing and the liner; an elongate body sized and configured to substantially fill the aperture; and a pressure relief valve in fluid communication with the volume of air; wherein the elongate body contains a heating element. In a related example, the elongate body may have a low friction surface. In a related example, the elongate body may be lubricated. In a further related example, the artificial vagina may have a hand pump. In a further related example, the elongate body may be component of a stand configured to receive the elongate housing and the liner. In a still further related example, the elongate housing may be at least 14 inches long. In a still further related example, the elongate housing may have an internal diameter of at least 3.5 inches. In a still further related example, the artificial vagina may have a thermometer in proximity to the elongate body. In a still further related example, the thermometer may be a thermocouple configured to regulate a heater. In a still further related example, the pressure relief valve may be a variable pressure relief valve.

Artificial vaginas described herein may, for example, comprise: an elongate housing having a first opening, a second opening and an internal volume; a liner located within the elongate housing wherein the liner is attached to the elongate housing; a volume of air between the elongate housing and the liner wherein the volume of air is at least 35° C. and wherein the volume of air has a gauge pressure of at least 5 mmHg; a thermometer in proximity to the liner; a relief valve in fluid communication with the volume of air; a positive pressure warm air supply in fluid communication with the volume of air; a temperature controller controlling the positive pressure warm air supply based on information from the thermometer and an exhaust port in fluid communication with the volume of air; wherein the volume of air fills greater than 70% of the internal volume and wherein the positive pressure warm air supply is configured to separate from the elongate housing and the liner. In a related example, the artificial vagina may have a hand pump. In a further related example, the elongate housing may be at least 14 inches long. In a further related example, the elongate housing may have an internal diameter of at least 3.5 inches. In a further related example, the thermometer may be a thermocouple in communication with a heater. In a still further related example, the artificial vagina may have a pressure relief valve in fluid communication with the volume of air.

The above-described embodiments have a number of independently useful individual features that have particular utility when used in combination with one another including combinations of features from embodiments described separately. There are, of course, other alternate embodiments which are obvious from the foregoing descriptions of the invention, which are intended to be included within the scope of the invention, as defined by the following claims.

What is claimed is:

1. An artificial vagina comprising:
a. an elongate housing having a first opening and a second opening;
b. a liner located within the elongate housing wherein the liner is attached to the elongate housing;
c. a volume of air between the elongate housing and the liner wherein the volume of air is at least 35° C. and wherein the volume of air has a gauge pressure of at least 5 mmHg;
d. an aperture centrally located within the elongate housing and the liner;
e. an elongate body sized and configured to substantially fill the aperture; and
f. a pressure relief valve in fluid communication with the volume of air;
g. wherein the elongate body contains a heating element.

2. The artificial vagina of claim 1 wherein the elongate body comprises a low friction surface.

3. The artificial vagina of claim 1 wherein the elongate body is lubricated.

4. The artificial vagina of claim 1 further comprising a hand pump.

5. The artificial vagina of claim 1 wherein the elongate body is a component of a stand configured to receive the elongate housing and the liner.

6. The artificial vagina of claim 1 wherein the elongate housing is at least 14 inches long.

7. The artificial vagina of claim 1 wherein the elongate housing has an internal diameter of at least 3.5 inches.

8. The artificial vagina of claim 1 further comprising a thermometer in proximity to the elongate body.

9. The artificial vagina of claim 1 wherein the thermometer is a thermocouple in communication with a heater.

10. The artificial vagina of claim 1 wherein the pressure relief valve is a variable pressure relief valve.

11. An artificial vagina comprising:
a. an elongate housing having a first opening, a second opening and an internal volume;
b. a liner located within the elongate housing wherein the liner is attached to the elongate housing;
c. a volume of air between the elongate housing and the liner wherein the volume of air is at least 35° C. and wherein the volume of air has a gauge pressure of at least 5 mmHg;
d. a thermometer in proximity to the liner;
e. a relief valve in fluid communication with the volume of air;
f. a positive pressure warm air supply in fluid communication with the volume of air;
g. a temperature controller controlling the positive pressure warm air supply based on information from the thermometer and
h. an exhaust port in fluid communication with the volume of air;
i. wherein the volume of air fills greater than 70% of the internal volume and
j. wherein the positive pressure warm air supply is configured to separate from the elongate housing and the liner.

12. The artificial vagina of claim 11 further comprising a hand pump.

13. The artificial vagina of claim 11 wherein the elongate housing is at least 14 inches long.

14. The artificial vagina of claim 11 wherein the elongate housing has an internal diameter of at least 3.5 inches.

15. The artificial vagina of claim 11 wherein the thermometer is a thermocouple configured to regulate a heater.

16. The artificial vagina of claim 11 further comprising a pressure relief valve in fluid communication with the volume of air.
